(19) United States
(12) Patent Application Publication
(10) Pub. No.: US 2023/0398042 A1
(43) Pub. Date: Dec. 14, 2023

(54) COSMETIC COMPOSITIONS AND USES THEREOF

(71) Applicants: william Robert Gately, Montclair, NJ (US); Robyn Kolas, Ellicott City, MD (US); Alyssa SCHNEIDER, Randolph, NJ (US); Coty Inc., New York, NY (US)

(72) Inventors: William Robert Gately, Montclair, NJ (US); Robyn Kolas, Ellicott City, MD (US); Alyssa Schneider, Randolph, NJ (US)

(21) Appl. No.: 18/248,048

(22) PCT Filed: Aug. 31, 2021
(86) PCT No.: PCT/US2021/048529

§ 371 Date: Apr. 5, 2023

Related U.S. Application Data
(60) Provisional application No. 63/088,088, filed on Oct. 6, 2020.
(60) Provisional application No. 63/157,247, filed on Mar. 5, 2021.

(51) Int. Cl. A61K 8/06; A61K 8/37; A61K 8/891; A61K 8/44; A61K 8/19; A61Q 1/12; A61Q 17/04; A61K 8/86

(52) U.S. Cl. CPC A61K 8/064; A61K 8/37; A61K 8/891; A61K 8/44; A61K 8/19; A61Q 1/12; A61Q 17/04; A61K 8/86; A61K 2800/43

(57) ABSTRACT

Various aspects according to the present disclosure relate to a water-in-oil emulsion composition. The composition includes a continuous oil phase. The continuous oil phase includes an oleochemical component, a surface-treated pigment component, and a surfactant component. The composition further includes a dispersed water phase including water and at least one solubilized material.

### CROSS-REFERENCE TO RELATED APPLICATIONS

This application claims the priority of U.S. Provisional Patent Application 63/088,088 titled “COSMETIC COMPOSITIONS AND USES THEREOF”, filed on Oct. 6, 2020, the contents of which are hereby incorporated by reference. This application also claims the priority of U.S. Provisional Patent Application 63/157,247 titled “COSMETIC COMPOSITIONS AND USES THEREOF”, filed on Mar. 5, 2021, the contents of which are hereby incorporated by reference.

### BACKGROUND

Various cosmetic compositions are used by consumers to cover perceived imperfections, apply a desired color, or another purpose. For a product to be viable for consumer use, the product not only must function for its intended purpose, but it also should feel comfortable to the user.

### SUMMARY

Various aspects according to the present disclosure relate to a water-in-oil emulsion composition. The composition includes a continuous oil phase. The continuous oil phase includes an oleochemical component, a surface-treated pigment component, and a surfactant component. The composition further includes a dispersed water phase including water and at least one solubilized material.

Various aspects according to the present disclosure relate to a water-in-oil emulsion composition. The composition includes a continuous oil phase. The continuous oil phase includes an oleochemical component that includes a mixture of dicaprylyl carbonate and dicaprylyl ether, a surface-treated pigment component that includes comprising stearoyl glutamic acid-coated titanium dioxide, and a surfactant component that includes Triglycerol diisosterarate and glyceryl stearate; and Cetyl PEG/PPG 10/1 Dimethicone. The composition further includes a dispersed water phase including water and at least one solubilized material.

Various aspects according to the present disclosure relate to a method of making a water-in-oil emulsion composition. The composition includes a continuous oil phase. The continuous oil phase includes an oleochemical component, a surface-treated pigment component, and a surfactant component. The composition further includes a dispersed water phase including water and at least one solubilized material. The method includes mixing water with at least one of the oleochemical component, surface-treated pigment component; surfactant component.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

Various aspects according to the instant disclosure relate to a water-in-oil emulsion composition. The water-in-oil emulsion composition can take the form of a cosmetic composition (in some aspects, the cosmetic composition can be over-the-counter drug (OTC)). Non-limiting examples of cosmetic compositions can include a concealer, a foundation, a bb cream, a sunscreen, or a combination thereof. The water-in-oil emulsion includes a continuous oil phase and a dispersed water phase. The continuous oil phase can be characterized as being predominantly non-polar in its surface tension. The continuous oil phase and dispersed water phase can independently range from about 5 wt % to about 95 wt % of the water-in-oil emulsion, about 10 wt % to about 90 wt %, about 15 wt % to about 85 wt %, about 20 wt % to about 80 wt %, about 25 wt % to about 75 wt %, about 30 wt % to about 70 wt %, about 35 wt % to about 65 wt %, about 40 wt % to about 60 wt %, about 45 wt % to about wt %, less than, equal to, or greater than about, 5 wt %, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or about 95 wt %.

The continuous oil phase can include various components. For example, the continuous oil phase can include at least an oleochemical component, a surface-treated pigment component, and a surfactant component. The dispersed water phase includes a solubilized material at least partially dissolved in water. The solubilized material can include a salt.

The oleochemical component can be any compound or mixture of compounds that are derived from vegetable oils, animal oils, fats, or from petrochemical feedstocks though physico-chemical modifications. The oleochemical component can range from about wt % to about 75 wt % of the water-in-oil emulsion composition, about 15 wt % to about 40 wt %, less than, equal to, or greater than, 10 wt %, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 wt %.

The oleochemical component can include one or more compounds or constituents having a weight-average molecular weight in a range of from about 10 Daltons to about 600 Daltons, about 200 Daltons to about 400 Daltons, less than, equal to, or greater than about 10 Daltons, 50, 100, 150, 200, 250, 300, 350, 400, 450, 500, 550, or about 600 Daltons. Examples of suitable oleochemical compounds can include a fatty acid, a fatty acid methyl ester, a fatty alcohol, a fatty amine, a glycerol, or a mixture thereof. In some aspects, the oleochemical component can include dicaprylyl carbonate, dicaprylyl ether, or a mixture thereof. Where present as a mixture of dicaprylyl carbonate and dicaprylyl ether, the dicaprylyl carbonate and dicaprylyl ether are independently in a range of from about 10 wt % to about 90 wt % of oleochemical component, about 15 wt % to about 85 wt %, about 20 wt % to about 80 wt %, about 25 wt % to about 75 wt %, about 30 wt % to about 70 wt %, about 35 wt % to about 65 wt %, about 40 wt % to about 60 wt %, about 45 wt % to about 50 wt %, less than, equal to, or greater than about, 10 wt %, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 72, 73, 74, 75, 76, 77, 78, 79, 80, 81, 82, 83, 84, 85, 86, 87, 88, 89, or about 90 wt %. In some specific embodiments, the dicaprylyl carbonate and dicaprylyl ether are in each about 50 wt % of the oleochemical component.

The surface-treated pigment component can be present in a range of from about 5 wt % to about 15 wt % of the water-in-oil emulsion composition, about 7 wt % to about 13 wt %, less than, equal to, or greater than about 5 wt %, 6, 7, 8, 9, 10, 11, 12, 13, 14, or about 15 wt %. The surface-treated pigment component can include, mica, a metal oxide (e.g., titanium dioxide, iron oxide, zinc oxide, or chromium oxide, or mixtures thereof. As non-limiting examples, iron oxides can include iron oxide red, iron oxide yellow, iron oxide black. As a further non-limiting example chromium oxides can include chromium oxide green.

The surface of all or a portion of the individual pigments of the pigment components can be at least partially coated. Examples of suitable coatings include a lauroyl lysine coating, a silane coating, a methicone coating, a dimethicone coating, a stearoyl glutamic acid, or a combination thereof. As generally understood, a lauroyl lysine is an amino acid derivative, derived from natural product lysine a protein and lauric acid another natural coconut fatty acid. lauroyl lysine treatment creates hydrophobic system with enhanced dispersion, increased wear properties and make up with a wet feel on the skin. Pigment surfaces are smooth, flat and less fluffy than with silane treatment, thus easier to press. Lauroyl lysine coatings are lubricious, giving the substrate a smooth, moist feel. As generally understood, a silane coating is a deposition of coatings through the reaction of silanols with the pigment. By hydrolysis of “Si—OR” groups to silanol (Si—OH), alkyl silane can self-associate or hydrogen bond to the pigment surface, so stable bonds are formed between the pigment and the treating compound following curing. Silicone treated pigments disperse well in cyclomethicones. They have very low surface tension, so they have excellent hydrophobicity and better lipophilicity due to which they easily disperse in mineral oils, esters and silicone fluids. Pigments treated with alkyl silane are more hydrophobic than methicone treated pigments, wet better in commonly utilized cosmetic oils and have lower oil absorption. In hydrous compact formulations, the excellent wetting imparted by alkyl silane treatment allows incorporation of high pigment loads to achieve a “powdery” sensation upon application to the skin while maintaining a low melt viscosity for hot filling. As generally understood, a methicone coating is a polymethylhydrogen siloxane, which absorb traces of water from pigment surface and converts Si—H bond to Si—OH with liberation of small amount of hydrogen gas and upon curing form covalent Si-0 pigment chemical bonds. The coating formed is highly hydrophobic and not solubilized by common solvents. The pigments wet well in oils particularly silicone oils. Skin feel is somewhat dry with enhanced slip and spreadability. As generally understood, a dimethicone coating (also called polymethylsiloxane) is a coating bonded to a pigment or filler surface via the mechanism of hydrolysis, condensation and curing to a Si—O pigment linkage. Surface treated with dimethicone are quite hydrophobic and have good slip and more lubricious feel. It is useful in oil based systems, which may be used for anhydrous products.

On individual pigments, the coating can substantially coat 100% of the total surface area of the individual pigment. In some further aspects, the coating can coat about 5% to about 95% of the total surface area of the individual pigment about 10% of the total surface area to about 90% of the total surface area, about 15% of the total surface area to about % of the total surface area, about 20% of the total surface area to about 80% of the total surface area, about 25% of the total surface area to about 75% of the total surface area, about 30% of the total surface area to about 70% of the total surface area, about 35% of the total surface area to about 65% of the total surface area, about 40% of the total surface area to about 60% of the total surface area, about 45% of the total surface area to about 50% of the total surface area, less than, equal to, or greater than about, 5% of the total surface area, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or about 95% of the total surface area. In some aspects, at least a portion of the total number of pigments present may be substantially free of any coating. For example, when less than 100% of the total number of pigments include a coating about 5% to about 95% of the total number of pigments can include a coating, about 10% to about 90% of the total number of pigments can include a coating, about 15% to about % of the total number of pigments can include a coating, about 20% to about 80% of the total number of pigments can include a coating, about 25% to about 75% of the total number of pigments can include a coating, about 30% to about 70% of the total number of pigments can include a coating, about 35% to about 65% of the total number of pigments can include a coating, about 40% to about 60% of the total number of pigments can include a coating, about 45% to about 50% of the total number of pigments can include a coating, less than, equal to, or greater than about, 5% of the total number of pigments can include a coating, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or about 95%. Each pigment can be coated with the same material or a different portions of the pigments can be at least partially coated with different coatings. An example of a suitable coating is stearoyl glutamic acid.

In some examples in which the coating is stearoyl glutamic acid, the composition includes Triglycerol diisosterarate, glyceryl stearate, or a combination thereof. Solvents used in such a system can include an organic solvent or an aqueous solvent. Examples of organic solvents can include a substituted or unsubstitued (C1-C20)hydrocarbyl. The term “hydrocarbyl,” as used herein, refers to a molecule or functional group that includes carbon and hydrogen atoms. The term can also refer to a molecule or functional group that normally includes both carbon and hydrogen atoms but wherein all the hydrogen atoms are substituted with other functional groups. A hydrocarbyl group can be a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are positive integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The water-in-oil emulsion composition can further include a surfactant component. Where present, the surfactant component can be in a range of from about 0.5 wt % to about 80 wt % of the water-in-oil emulsion composition, about 1 wt % to about 6 wt %, less than, equal to, or greater than about, 0.5 wt %, 1, 2, 3, 4, 5, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 67, 68, 69, 70, 71, 72, 73, 74, 75, 76, 77, 78, 79, or about 80 wt %. Examples of suitable surfactants can include anionic surfactants, non-ionic surfactants, amphoteric surfactants, zwitterionic surfactants, cationic surfactants, and mixtures thereof.

Anionic surfactants may be selected from a group of salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, a-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; N-acyltaurates; and mixtures thereof. The alkyl or acyl radical of all of these various compounds, for example, comprises from 8 to 24 carbon atoms, and the aryl radical, for example, is chosen from phenyl and benzyl groups. Among the anionic surfactants, which can also be used, mention may also be made of fatty acid salts such as the salts of oleic, ricinoleic, palmitic and stearic acids, coconut oil acid or hydrogenated coconut oil acid; acyl lactylates in which the acyl radical comprises from 8 to carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

Suitable anionic surfactant(s) may comprise at least one anionic functional groups at their head selected from sulfate, sulfonate, phosphate and carboxylates.

Suitable alkyl sulfates include ammonium lauryl sulfate, sodium lauryl sulfate (sodium dodecyl sulfate, SLS, or SDS), and alkyl-ether sulfates, such as sodium laureth sulfate (sodium lauryl ether sulfate or SLES), and sodium myreth sulfate.

Further suitable anionic surfactants may include Docusate (dioctyl sodium sulfosuccinate), alkyl-aryl ether phosphate, alkyl ether phosphate, alkyl carboxylate, such as sodium stearate, sodium lauroyl sarcosinate, ammonium laureth sulfate, disodium lauryl sulfosuccinate, and sodium lauryl sulphoacetate.

Preferred anionic surfactants may be selected from a group of sodium laurylethersulfate, sodium laurethethersulfate, sodium dodecyl sulfate, ammonium laurethethersulfate, ammonium dodecyl sulfate, alkylbenzenesulfonate, and combinations thereof.

Non-ionic surfactant(s) may be selected from a group disclosure of lanolin alcohol, and polyoxyethylene ethers of fatty alcohols, and mixtures thereof. The non-ionic surfactant may be preferably ceteareth-n, wherein n is from 2 to 100, or from 10 to 30. When the one or more surfactants of the composition are non-ionic, precipitation of others ingredients of the composition can be prevented. Suitable nonionic surfactants are compounds that are well known (see, for example, in this respect “Handbook of Surfactants” by M. R. Porter, published by Blackie & Son (Glasgow and London), 1991, pp. 116-178).

The nonionic surfactants are more particularly chosen from monooxyalkylenated or polyoxyalkylenated, monoglycerolated or polyglycerolated nonionic surfactants. The oxyalkylene units are more particularly oxyethylene or oxypropylene units, or a combination thereof, such as POE/POP/PGE (INCI: Poloxamer 184) (trade name: Pluracare L64, BASF), preferably oxyethylene units.

Examples of oxyalkylenated nonionic surfactants that may be mentioned include:
oxyalkylenated (C₈-C₂₄)alkylphenols,
saturated or unsaturated, linear or branched, oxyalkylenated C₈-C₃₀ alcohols such as oxyethylenated cetylstearyl alcohol (33OE) or oleyl alcohol (10OE),
saturated or unsaturated, linear or branched, oxyalkylenated C₈-C₃₀ amides
esters of saturated or unsaturated, linear or branched, C₈-C₃₀ acids and of polyethylene glycols,
polyoxyethylenated esters of saturated or unsaturated, linear or branched, C₈-C₃₀ acids and of sorbitol,
saturated or unsaturated, oxyethylenated plant oils,
condensates of ethylene oxide and/or of propylene oxide, and mixtures thereof.

Monoglycerolated or polyglycerolated nonionic surfactants, monoglycerolated or polyglycerolated C₈-C₄₀ alcohols are preferably used. In particular, the monoglycerolated or polyglycerolated C₈-C₄₀ alcohols correspond to the following formula:

RO—[CH₂CH(CH₂0H)—O]ₘ—H

in which R represents a linear or branched C₈-C₄₀ and preferably C₈-C₃₀ alkyl or alkenyl radical, and m represents a number ranging from 1 to 30 and preferably from 1 to 10.

As examples of compounds that are suitable in the context of the disclosure, mention may be made of lauryl alcohol containing 4 mol of glycerol (INCI name: Polyglyceryl-4 Lauryl Ether), Triglycerol diisosterarate and glyceryl stearate, lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

In some examples that include triglycerol diisosterarate and glyceryl stearate, the composition is free of Cetyl PEG/PPG 10/1 Dimethicone.

Among the monoglycerolated or polyglycerolated alcohols, it is more particularly preferred to use the C₈-C₁₀ alcohol containing 1 mol of glycerol, the C₁₀-C₁₂ alcohol containing 1 mol of glycerol and the C₁₂ alcohol containing 1.5 mol of glycerol.

Other surfactants can include Cetyl PEG/PPG 10/1 Dimethicone.

In some aspects, the salt in the dispersed water phase can include sodium chloride, magnesium sulfate, or mixtures thereof.

In some aspects, the continuous oil phase, dispersed water phase, or both can include a chelant or preservative component. Where present, the preservative (or a mixture of preservatives) is in a range of from about 0.1 wt % to about 10 wt % of the water-in-oil emulsion composition, about 0.5 wt % to about 9.5 wt %, about 1 wt % to about 9 wt % about 1.5 wt % to about 8.5 wt %, about 2 wt % to about 8 wt %, about 2.5 wt % to about 7.5 wt %, about 3 wt % to about 7 wt %, about 3.5 wt % to about 6.5 wt %, about 4 wt % to about 6 wt %, about 4.5 wt % to about 5.5 wt %, less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, or about 10 wt %. Classes of preservatives can include a sodium salt, a potassium salt, a lactobacillus ferment, a yeast ferment, an aromatic alcohol, a linear chain alcohol, or a mixture thereof.

Specific examples of preservatives can include one or more aminocarboxylic acid preservatives comprising one or more carboxylic acid moieties (—COOH) and one or more nitrogen atoms. The one or more aminocarboxylic acid preservatives may be selected from a group of diethylenetriamine pentaacetic acid (DTPA), diethylenetriamine-N,N′,N″-polyacids, ethylenediamine disuccinic acid (EDDS), ethylenediamine-N,N′-diglutaric acid (EDDG), 2-hydroxypropylenediamine-N-N′-disuccinic acid (HPDDS), glycinamide-N,N′-disuccinic acid (GADS), ethylenediamine-N-N′-diglutaric acid (EDDG), 2-hydroxypropylenediamine-N-N′-disuccinic acid (HPDDS), ethylenediaminetetraacetic acid (EDTA), ethylenedicysteic acid (EDC), ethylenediamine-N-N′-bis(ortho-hydroxyphenyl acetic acid) (EDDHA), diaminoalkyldi(sulfosuccinic acids) (DDS), N,N′-bis(2-hydroxybenzyl)ethylenediamine-N,N′-diacetic acid (HBED), ethylene diamine tri(methylene phosphonate), hexamethylene diamine tetra (methylene phosphonate), their salts thereof, and mixtures thereof.

Alternatively, the one or more aminocarboxylic acid preservatives may be selected from a group of iminodiacetic acid derivatives such as N-2-hydroxyethyl N,N diacetic acid or glyceryl imino diacetic acid, iminodiacetic acid-N-2-hydroxypropyl sulfonic acid and aspartic acid N-carboxymethyl N-2-hydroxypropyl-3-sulfonic acid, β-alanine-N,N′-diacetic acid, aspartic acid-N,N′-diacetic acid, aspartic acid-N-monoacetic acid and iminodisuccinic acid preservatives, ethanoldiglycine acid, dipicolinic acid, 2-phosphonobutane-1,2,4-tricarboxylic acid, their salts thereof, their derivatives thereof, and mixtures thereof.

The one or more preservatives may be one or more aminophosphonic acid preservatives comprising an aminophosphonic acid moiety (—PO₃H₂) or its derivative PO₃R₂, wherein R₂ is a C₁ to C₆ alkyl or aryl radical and salts thereof.

The one or more aminophosphonic acid preservatives may be selected from a group of aminotri-(1-ethylphosphonic acid), ethylene-diaminetetra-(1-ethylphosphonic acid), aminotri-(1-propylphosphonic acid), aminotri-(isopropylphosphonic acid), their salts thereof, and mixtures thereof; alternatively aminotri-(methylenephosphonic acid), ethylene-diamine-tetra-(methylenephosphonic acid) (EDTMP) and diethylene-triamine-penta-(methylenephosphonic acid) (DTPMP), their salts thereof, their derivatives thereof, and mixtures thereof.

Suitable alternative preservatives include, but are not limited to: polyethyleneimines, polyphosphoric acid preservatives, etidronic acid, methylglycine diacetic acid, N-(2-hydroxyethyl)iminodiacetic acid, minodisuccinnic acid, N,N-Dicarboxymethyl-L-glutamic acid, N-lauroyl-N,N′,N″-ethylenediamine diacetic acid, their salts thereof, their derivatives thereof, and mixtures thereof.

Other various preservatives may also be contemplated, including the amino phosphonates, available as Dequest™ from Monsanto, the nitriloacetates, the hydroxyethyl-ethylene triamines and the like which are known for such use. Suitable preservatives for use herein may include organic phosphonates, such as the amino alkylene poly (alkylene phosphonates), alkali metal ethane 1-hydroxy disphosphonates and nitrilo trimethylene phosphonates.

In some aspects, the water-in-oil emulsion composition can include an oil absorber component. The oil absorber can be located in the oil phase. Where present, the oil absorber is in a range of from about 0.1 wt % to about 10 wt % of the water-in-oil emulsion composition, about 0.5 wt % to about 7 wt %, about 1 wt % to about 6 wt %, about 2 wt % to about 5 wt %, about 3 wt % to about 4 wt %, less than, equal to, or greater than about 0.1 wt %, 1, 1.5, 2, 2.5, 3, 3.5, 4, 4.5, 5, 5.5, 6, 6.5, 7, 7.5, 8, 8.5, 9, 9.5, or about 10 wt %. Examples of suitable oil absorbers can include a silica particle, a modified starch, a modified talc, a modified clay, or a mixture thereof.

The oil absorber can be present as a collection of particles. An average particle size of the particles can independently be in a range of from about 0.1 nm to about 15 microns, about 4 microns to about 13 microns, about 6 microns to about 11 microns, less than, equal to, or greater than about 0.1 nm, 1 nm, 2 microns, 3 microns, 4 microns, 5 microns, 6 microns, 7 microns, 8 microns, 9 microns, 10 microns, 11 microns, 12 microns, 13 microns, 14 microns, or about 15 microns. A surface area of the individual particles, can independently be in a range of from about 100 m²/g to about 1000 m²/g, about 200 m²/g to about 300 m²/g, less than, equal to, or greater than about 100 m²/g, 120, 140, 160, 180, 200, 220, 240, 260, 280, 300, 320, 340, 360, 380, 400, 450, 500, 550, 600, 650, 700, 750, 800, 850, 900, 950, or about 1000 m²/g.

One function of the oil absorber can be to absorb sebum oil. Sebum oil is a natural oil that is constantly produced by glands, for example, on the scalp. Sebum oil can make hair look greasy. Certain oil absorbers such as silica or others described herein can be effective at absorbing sebum, but can create a perceived draggy or heavy feel for the user. However, using the dicaprylyl ether and dicaprylyl carbonate described herein can create a smooth or pleasant feel for the user that mitigates the negative feel associated with these oil abosrbers. Thus, the compositions herein can satisfactorily absorb sebum while counteracting the negative feel associated with oil absorbers.

According to various aspects, the water-in-oil emulsion composition is substantially free of cyclopentasiloxane. For example, the water-in-oil emulsion composition includes less than 5 wt %, less than 4 wt %, less than 3 wt %, less than 2 wt %, less than 1 wt %, less than 0.5 wt %, less than, 0.3 wt %, less than 0.1 wt %, less than 0.01 wt %, less than 0.001 wt %. In some examples, the water-in-oil composition emulsion includes 0 wt % cyclopentasiloxane.

According to various aspects, the water-in-oil emulsion composition can include a humectant component. Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, butylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin, butylene glycol, sodium hyaluronate, glycerin and mixtures thereof. When present, the humectant component is present in a range of from about 0.1 wt % to about 10 wt % of the composition, about 2.5 wt % to about 5 wt %, less than, equal to, or greater than, about 0.1 wt %, 0.5, 1, 1.5, 2, 2.5, 3, 3.5, 4, 4.5, 5, 5.5, 6, 6.5, 7, 7.5, 8, 8.5, 9, 9.5, or about 10 wt %.

According to various aspects, the water-in-oil emulsion composition can include a vitamin component. The vitamin component can function as a skin-care active. The vitamin component can include a vitamin or a derivative of vitamin. For example, the vitamin component can include niacinamide, panthenol, tocopherol, vitamin C, vitamin A, derivatives thereof, or a mixture thereof. When present, the vitamin component is present in a range of from about 0.1 wt % to about 10 wt % of the composition, about 1 wt % to about 8 wt %, less than, equal to, or greater than, about 0.1 wt %, 0.5, 1, 1.5, 2, 2.5, 3, 3.5, 4, 4.5, 5, 5.5, 6, 6.5, 7, 7.5, 8, 8.5, 9, 9.5, or about 10 wt %.

According to various aspects, the water-in-oil emulsion composition can include an antioxidant component. The vitamin component can include tocopherol, vitamin C, derivatives thereof, or a mixture thereof. When present, the antioxidant component is present in a range of from about 0.1 wt % to about 10 wt % of the composition, about 0.5 wt % to about 2 wt %, less than, equal to, or greater than, about 0.1 wt %, 0.5, 1, 1.5, 2, 2.5, 3, 3.5, 4, 4.5, 5, 5.5, 6, 6.5, 7, 7.5, 8, 8.5, 9, 9.5, or about 10 wt %.

According to various aspects, the water-in-oil emulsion composition can include a film former component. The film former component can function to help the composition to form a substantially even film on a user's skin. The film former component can include polyvinylpyrrolidone, trimethylsiloxysilcate, an acrylate polymer, or a mixture thereof. When present, the film former component is present in a range of from about 0.5 wt % to about 20 wt % of the composition, about 1 wt % to about 5 wt %, less than, equal to, or greater than, about 0.5 wt %, 1, 1.5, 2, 2.5, 3, 3.5, 4, 4.5, 5, 5.5, 6, 6.5, 7, 7.5, 8, 8.5, 9, 9.5, 10, 11, 11.5, 12, 12.5, 13, 13.5, 14, 14.5, 15, 15.5, 16, 16.5, 17, 17.5, 18, 18.5, 19, 19.5 or about 20 wt %.

According to various aspects, the water-in-oil emulsion composition can include a sunscreen component. The sunscreen component can function to help the composition to provide the benefit to the user of absorbing at least some ultraviolet radiation. The sunscreen component can include zinc oxide (present at about 15 wt % to about 30 wt % of the composition), titanium dioxide (present at about 15 wt % to about 30 wt % of the composition), octinoxate (present at about 15 wt % to about 30 wt % of the composition), ensulizole (present at about 5 wt % to about 15 wt % of the composition), ethylhexyl triazone(present at about 15 wt % to about 30 wt % of the composition), octyl salicylate(present at about 2 wt % to about 7 wt % of the composition), butyl methoxydibenzoylmethane(present at about 5 wt % to about 20 wt % of the composition), bisethylhexyloxyphenol methoxyphenyl triazine(present at about 5 wt % to about 15 wt % of the composition), isoamyl-p-methoxycinnamate(present at about 5 wt % to about 15 wt % of the composition), diethylhexyl butamido triazone(present at about 5 wt % to about 15 wt % of the composition), or a mixture thereof.

According to various aspects, the water-in-oil emulsion composition can include an optical modifier component. The optical modifier component can function to control the refractive index of the composition and, in turn, help the composition to achieve a desired finish. For example, the optical modifier can help to achieve a glossy or matte finish. The optical modifier component comprises a dimethicone/vinyl dimethicone crosspolymer, a C30-45 alkyldimethylsilyl polypropylsilsesquioxane, or a mixture thereof. When present, the optical modifier component is present in a range of from about 0.1 wt % to about 20 wt % of the composition, about 0.5 wt % to about 10 wt %, less than, equal to, or greater than, about wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or about 20 wt %.

In the water-in-oil emulsion composition the total solids content of the water-in-oil emulsion composition is in a range of from about 1 wt % to about 40 wt %, about 5 wt % to about 25 wt %, less than, equal to, or greater than about 1 wt %, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 wt %. In some aspects, a major portion of the total solids content is the surface-treated pigment component. For example, the surface-treated pigment component can account for about 50 wt % to about 100 wt % of the total solids content, about 70 wt % to about wt %, less than, equal to, or greater than about 50 wt %, 55, 60, 65, 70, 75, 80, 85, 90, 95, or about 100 wt %.

The mixture of components found in the water-in-oil emulsion composition can result in the composition having a desired surface tension. In various aspects, it is desirable to have the surface tension of the water-in-oil emulsion composition to be substantially equivalent to a surface tension value of mammalian skin-human skin in particular. The surface energy of human skin is typically in a range of from about 28 mJ/m² to about 54 mJ/m². Acceptable surface tension values of the water-in-oil emulsion composition are in a range of from about 15 mJ/m² to about 50 mJ/m², about 20 mJ/m² to about 34 mJ/m², less than, equal to, or greater than about 15 mJ/m², 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mJ/m². Having the surface tension of the water-in-oil composition be substantially similar to that of mammalian skin can help to improve the degree to which the water-in-oil composition is able to spread on the mammalian skin. In some aspects, it is desirable for the surface tension of the water-in-oil composition to be less than the surface energy of the mammalian skin.

The surface tension of the water-in-oil composition is a result of the individual surface tension values of the compositions components. Two components that have a particular effect on the surface tension value of the water-in-oil composition are the surface energy values of the surface-treated pigment component and the surface tension value of the oleochemical component. Critically, the surface tension of the oleochemical component is greater than the surface energy of the surface-treated pigment component. The surface tension values of the surface-treated pigment component and the oleochemical components are average measurements of the constituents of the surface-treated pigment component and the constituents of the oleochemical component, respectively.

According to various aspects, a surface energy of the surface-treated pigment component is in a range of from about 1 mJ/m² to about 20 mJ/m², about 5 mJ/m² to about 15 mJ/m², less than, equal to, or greater than about 1 mJ/m², 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 mJ/m². Additionally, according to various aspects, the surface tension of the oleochemical component is in a range of from about 25 mN/m to about mN/m, about 27 mN/m to about 29 mN/m, less than, equal to, or greater than about 25 mN/m, 25.5, 26, 26.5, 27, 27.5, 28, 28.5, 29, 29.5, or about 30 mN/m.

A closed cup flashpoint of the water-in-oil emulsion composition is greater than 73° C., greater than about 100° C., in a range of from about 25° C. to about 200° C., about 50° C. to about 150° C., about 70° C. to about 110° C., less than equal to, or greater than about ° C., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or about 200° C. Achieving these flash points is desirable in conjunction with the surface tension values described herein because it allows for the water-in-oil emulsion composition to spread on the user's skin to a desired degree while not substantially evaporation before a desired time. Additionally, the combined flash point and surface tension of the water-in-oil emulsion composition results in the composition having a good spreading rate. The spreading rate can be function of the spreading rate (mm²/10 min) of the oleochemical component, which is in a range of from about 1000 to about 3000 about 1200 to about 2000 mm²/10 min, less than, equal to, or greater than about 1000, 1100, 1200, 1300, 1400, 1500, 1600, 1700, 1800, 1900, 2000, 2100, 2200, 2300, 2400, 2500, 2600, 2700, 2800, 2900, or about 3000 mm²/10 min. The spreading rate is determined as the area measured in mm 2, which has a nearly point-like applied to a horizontal surface liquid after 10 minutes dwell time on this surface.

The water-in-oil emulsion composition is capable of achieving a contrast ratio (unitless) in a range of from about 0.5 to about 1, about 0.65 to about 1, less than, equal to, or greater than about 0.5, 0.55, 0.6, 0.65, 0.7, 0.75, 0.8, 0.85, 0.9, 0.95, or about 1. The contrast ratio can generally be understood to refer to a property of the water-in-oil emulsion composition, defined as the ratio of the luminance of the brightest color to that of the darkest color that the composition is capable of producing. Additionally, a coverage quotient of the water-in-oil emulsion composition can be in a range of from about 0.3 to about 1, about 0.8 to about 1, less than, equal to, or greater than about 0.5, 0.55, 0.6, 0.65, 0.7, 0.75, 0.8, 0.85, 0.9, or about 1. The coverage quotient can generally be understood to refer to the degree that the composition is able to spread over a desired area and provide an acceptable degree of coverage. Unexpectantly, it was found that water-in-oil emulsion compositions including the constituents described herein had equivalent or superior properties with respect to at least the contrast ratio and coverage quotient of a comparative water-in-oil emulsion that includes cyclopentasiloxane. Therefore, the instant disclosure describes a water-in-oil emulsion composition that can effectively replace those including cyclopentasiloxane.

Additionally, the water-in-oil emulsion compositions are able to provide a substantially non-greasy feel, non-oily feel, or both as determined according to the sensory analysis protocol in the Examples. These characteristics are also relevant to the composition's ability to increase its long-wear characteristics. A transfer failure mechanism of a composition can generally relate to the tendency of the composition to be removed from a user's skin through abrasion. Abrasion can be understood to relate to the wearing away of the composition as a result of an applied friction force. This can be counteracted, to some extent, by addition of film formers such as polyvinylpyrrolidone or trimethylsilyl. However these film formers are quite strong and can be rigid, both of which can cause discomfort to a user. A less rigid film former provides substantially less resistance to abrasion. However, the other components of the composition described herein such as the dicarprylyl ether and dicaprylyl carbonate can mitigate the negative feel characteristics of the rigid film formers. This allows for the compositions to be reasonably comfortable and resistant to abrasion, therefore, increasing the long-wear characteristics of the composition.

Additionally, the improved feel described herein with respect to the compositions may be at least partially attributed to the production of foams. Foams are understood to include a mass of small bubbles formed on or in a liquid. Bubbles formed during foaming reduce the mass per unit of volume of the composition. This can result in a lighter product overall. However, forming a foam in with the oils and polymers described herein in conjunction with air is not an expected result. Foam is far more likely to form with water, air, and a surfactant. The unexpected ability of the instant composition to be able to form a foam with the oils and polymers described herein can help the composition to feel relatively light and thus contribute to the improved feel characteristics described herein.

The water-in-oil emulsion composition was prepared by separately combining the constituents of the continuous oil phase and dispersed water phase. The respective constituents are mixed to form respective pre-emulsion solutions. The pre-emulsion solutions are then combined and mixed to form the water-in-oil emulsion.

In some examples, instead of acting as a surfactant, triglycerol diisosterarate, glyceryl stearate, or a mixture thereof can function as an emulsification system. In such examples, the emulsification system can be combined with any of the aforementioned components.

Additionally in some examples, the compositions described herein can include dicaprylyl ether, dicaprylyl carbonate, or a mixture thereof. The dicaprylyl ether, dicaprylyl carbonate, or the mixture thereof can be combined with any of the aforementioned components.

### Exemplary Aspects.

The following exemplary aspects are provided, the numbering of which is not to be construed as designating levels of importance:

Aspect 1 provides a water-in-oil emulsion composition comprising:
a continuous oil phase comprising:
   an oleochemical component;
   a surface-treated pigment component; and
   a surfactant component; and
a dispersed water phase comprising of water and at least one solubilized material.

Aspect 2 provides the water-in-oil emulsion composition of Aspect 1, wherein the oleochemical component is in a range of from about 10 wt % to about 75 wt % of the water-in-oil emulsion composition.

Aspect 3 provides the water-in-oil emulsion composition of Aspect 1, wherein the oleochemical component comprises Triglycerol diisosterarate and glyceryl stearate; Cetyl PEG/PPG 10/1 Dimethicone, or a mixture thereof.

Aspect 4 provides the water-in-oil emulsion composition of Aspect 3, wherein Triglycerol diisosterarate and glyceryl stearate; Cetyl PEG/PPG 10/1 Dimethicone are independently in a range of from about 10 wt % to about 90 wt % of oleochemical component.

Aspect 5 provides the water-in-oil emulsion composition of any one of Aspects 1-4, wherein the oleochemical component comprises a plurality of constituents independently having a molecular weight in a range of from about 10 Daltons to about 600 Daltons.

Aspect 6 provides the water-in-oil emulsion composition of any one of claims 1-5, wherein the surface-treated pigment component is in a range of from about 1 wt % to about 25 wt % of the water-in-oil emulsion composition.

Aspect 7 provides the water-in-oil emulsion composition of any one of Aspects 1-6, wherein the surface-treated pigment component comprises a metal oxide.

Aspect 8 provides the water-in-oil emulsion composition of any one of Aspects 1-7, wherein at least a portion of individual surface-treated pigments of the surface-treated pigment component are at least partially coated with stearoyl glutamic acid.

Aspect 9 provides the water-in-oil emulsion composition of any one of Aspects 1-8, wherein the surfactant component comprises a glycerol, an alkyl-modified dimethicone, or a mixture thereof.

Aspect 10 provides the water-in-oil emulsion composition of any one of claims 1-9, wherein the surfactant component is in a range of from about 0.5 wt % to about 80 wt % of the water-in-oil emulsion composition.

Aspect 11 provides the water-in-oil emulsion composition of any one of claims 1-10, wherein the surfactant component is in a range of from about 1 wt % to about 6 wt % of the water-in-oil emulsion composition.

Aspect 12 provides the water-in-oil emulsion composition of any one of Aspects 1-11, further comprising a preservative component in the dispersed water phase, oil phase, or both.

Aspect 13 provides the water-in-oil emulsion composition of any one of Aspects 1-12, wherein a total solids content of the water-in-oil emulsion composition is in a range of from about 1 wt % to about 40 wt %.

Aspect 14 provides the water-in-oil emulsion composition of any one of Aspects 1-13, wherein a surface tension of the water-in-oil emulsion composition is in a range of from about 15 mN/m to about 50 mN/m.

Aspect 15 provides the water-in-oil emulsion composition of any one of Aspects 1-14, wherein a surface energy of the surface-treated pigment component is in a range of from about 1 mJ/m² to about 20 mJ/m².

Aspect 16 provides the water-in-oil emulsion composition of any one of Aspects 1-15, wherein a surface tension of the oleochemical component is greater than a surface energy of the surface-treated pigment component.

Aspect 17 provides the water-in-oil emulsion composition of any one of Aspects 1-16, further comprising an oil absorber component in the oil phase.

Aspect 18 provides the water-in-oil emulsion composition of Aspect 17, wherein average particle sizes for of individual particles are independently in a range of from nm and 15 microns.

Aspect 19 provides the water-in-oil emulsion composition of any one of Aspects 1-18, wherein the composition comprises a concealer, a foundation, a bb cream, a sunscreen, or a combination thereof.

Aspect 20 provides the water-in-oil emulsion composition of any one of Aspects 1-19, wherein a contrast ratio of the water-in-oil emulsion composition is in a range of from about 0.65 to about 1.

Aspect 21 provides the water-in-oil emulsion composition of any one of Aspects 1-20, wherein a coverage quotient of the water-in-oil emulsion composition is in a range of from about 0.5 to about 1.

Aspect 22 provides the water in oil emulsion composition of any one of Aspects 1-21, wherein the composition further comprises a humectant component.

Aspect 23 provides the water in oil emulsion composition of any one of Aspects 1-22, wherein the composition further comprises a film former component.

Aspect 24 provides the water in oil emulsion composition of any one of Aspects 1-23, wherein the composition further comprises a sunscreen component.

Aspect 25 provides the water-in-oil emulsion of Aspect 24, wherein the sunscreen component is present in a range of from about 0.1 wt % to about 30 wt % of the composition.

Aspect 26 provides a water-in-oil emulsion composition comprising:
a continuous oil phase comprising:
   an oleochemical component comprising Triglycerol diisosterarate and glyceryl stearate; Cetyl PEG/PPG 10/1 Dimethicone, or a mixture thereof;
   a surface-treated pigment component comprising stearoyl glutamic acid-coated titanium dioxide; and
   a dispersed water phase comprising at least water

Aspect 27 provides a method of making the water-in-oil emulsion composition of any one of Aspects 1-26, the method comprising:
mixing water with at least one of the oleochemical component, surface-treated pigment component; surfactant component.

Aspect 28 provides the water-in-oil composition of any one of Aspects 1-27, further comprising a plurality of foam bubbles distributed about the composition.

1. A water-in-oil emulsion composition comprising:
a continuous oil phase comprising:
an oleochemical component;
a surface-treated pigment component; and
a surfactant component; and
a dispersed water phase comprising
of water and at least one solubilized material.

2. The water-in-oil emulsion composition of claim 1, wherein the oleochemical component is in a range of from about 10 wt % to about 75 wt % of the water-in-oil emulsion composition.

3. The water-in-oil emulsion composition of claim 1, wherein the oleochemical component comprises Triglycerol diisosterarate and glyceryl stearate; Cetyl PEG/PPG 10/1 Dimethicone, or a mixture thereof.

4. The water-in-oil emulsion composition of claim 3, wherein Triglycerol diisosterarate and glyceryl stearate; Cetyl PEG/PPG 10/1 Dimethicone are independently in a range of from about 10 wt % to about 90 wt % of oleochemical component.

5. The water-in-oil emulsion composition of claim 1, wherein the oleochemical component comprises a plurality of constituents independently having a molecular weight in a range of from about 10 Daltons to about 600 Daltons.

6. The water-in-oil emulsion composition of claim 1, wherein the surface-treated pigment component is in a range of from about 1 wt % to about 25 wt % of the water-in-oil emulsion composition.

7. The water-in-oil emulsion composition of claim 1, wherein the surface-treated pigment component comprises a metal oxide.

8. The water-in-oil emulsion composition of claim 1, wherein at least a portion of individual surface-treated pigments of the surface-treated pigment component are at least partially coated with stearoyl glutamic acid.

9. The water-in-oil emulsion composition of claim 1, wherein the surfactant component comprises a glycerol, an alkyl-modified dimethicone, or a mixture thereof.

10. The water-in-oil emulsion composition of claim 1, wherein the surfactant component is in a range of from about 0.5 wt % to about 80 wt % of the water-in-oil emulsion composition.

11. The water-in-oil emulsion composition of claim 1, wherein the surfactant component is in a range of from about 1 wt % to about 6 wt % of the water-in-oil emulsion composition.

12. The water-in-oil emulsion composition of claim 1, further comprising a preservative component in the dispersed water phase, oil phase, or both.

13. The water-in-oil emulsion composition of claim 1, wherein a total solids content of the water-in-oil emulsion composition is in a range of from about 1 wt % to about 40 wt %.

14. The water-in-oil emulsion composition of claim 1, wherein a surface tension of the water-in-oil emulsion composition is in a range of from about 15 mN/m to about 50 mN/m.

15. The water-in-oil emulsion composition of claim 1, wherein a surface energy of the surface-treated pigment component is in a range of from about 1 mJ/m² to about 20 mJ/m².

16. The water-in-oil emulsion composition of claim 1, wherein a surface tension of the oleochemical component is greater than a surface energy of the surface-treated pigment component.

17. The water-in-oil emulsion composition of claim 1, further comprising an oil absorber component in the oil phase.

18. The water-in-oil emulsion composition of claim 17, wherein average particle sizes for of individual particles are independently in a range of from 0.1 nm and 15 microns.

19. The water-in-oil emulsion composition of claim 1, wherein the composition comprises a concealer, a foundation, a bb cream, a sunscreen, or a combination thereof.

20. (canceled)

21. (canceled)

22. (canceled)

23. (canceled)

24. (canceled)

25. (canceled)

26. A water-in-oil emulsion composition comprising:
a continuous oil phase comprising:
an oleochemical component comprising Triglycerol diisosterarate and glyceryl stearate; Cetyl PEG/PPG 10/1 Dimethicone, or a mixture thereof;
a surface-treated pigment component comprising stearoyl glutamic acid-coated titanium dioxide; and
a dispersed water phase comprising at least water

27. (canceled)

28. (canceled)
